(19) United States
(12) Patent Application Publication
(10) Pub. No.: US 2011/0004074 A1
(43) Pub. Date: Jan. 6, 2011

(54) PROTECTIVE GLOVE WITH WIRELESS PHYSIOLOGICAL SENSOR

(75) Inventors: Arunraj V, Coimbatore (IN); VigneshKumar Natesan, Madurai (IN); Yuvanya G, Salem (IN); Abarna Shanmugasundaram, Chennai (IN)

Correspondence Address: HONEYWELL/HUSCH;Patent Services, 101 Columbia Road, P.O.Box 2245, Morrlstown, NJ, 07962, US

(73) Assignee: Honeywell International Inc., Morristown, NJ (US)

(21) Appl. No.: 12/497,849

(22) Filed: Jul. 6, 2009

(51) Int. Cl. A61B 5/02; A61B 5/145; A61B 5/01; A61B 5/021; A61B 5/024; A41D 19/00

(52) U.S. Cl. 600/301; 600/309; 600/549; 600/485; 600/500; 2/159

(57) ABSTRACT

A multi-finger protective glove carries one or more physiological sensors in respective fingers. An elastomeric sleeve is positioned in a respective sensor carrying finger to maintain the sensor in proper position relative to a user's finger inserted into the glove, adjacent to at least one of the sensors. The sensor can be attached to a distal end of the sleeve, adjacent an end of the finger of the glove.

### FIELD

The invention pertains to safety equipment for use by first responders, such as firefighters or hazardous materials control personnel. More particularly, the invention pertains to gloves usable by such individuals which can feedback physiological information to other local personnel, for example situation commanders, as to the health of the respective first responder.

### BACKGROUND

Various types of protective gloves usable by first responders are known. One such glove has been disclosed in published US Patent Application No. 2008/0263745 A1 entitled, “Protective Glove” published Oct. 30, 2008 and owned by the assignee hereof. The '745 published application is hereby incorporated herein by reference.

While quality protective leggings, coats and gloves as noted above are available to protect first responders in dangerous environmental situations, such as fires or the like, they provide no information as to the health or condition of the respective first responder. While such individuals may work in groups or teams, the group members are going to be occupied with addressing the subject environmental condition and will usually not have time to monitor one or another's physiological condition. The safety of such persons in the presence of dangerous conditions is an important consideration.

Thus it would be desirable to be able to extract physiological information in an unobtrusive way from such individuals and make it available to supervisory personnel who could direct a person exhibiting adverse physiological symptoms to leave the location of the fire or other hazardous condition and report for treatment.

### BRIEF DESCRIPTION OF THE DRAWINGS

FIG. 1 is an overall diagram of an apparatus which embodies the invention;

FIG. 2 is a diagram of a glove in accordance with the invention; and

FIG. 3 is a side sectional view taken along plane 3-3 of FIG. 2.

### DETAILED DESCRIPTION

While embodiments of this invention can take many different forms, specific embodiments thereof are shown in the drawings and will be described herein in detail with the understanding that the present disclosure is to be considered as an exemplification of the principles of the invention, as well as the best mode of practicing same, and is not intended to limit the invention to the specific embodiment illustrated.

In one embodiment, a protective glove can incorporate a physiological sensor such as a pulse oximeter such as a CO-oximeter, blood pressure sensor, temperature sensor, pulse rate sensor or the like without limitation. The respective sensor can be wirelessly coupled to a displaced, or remote, monitoring unit. Individuals at the monitoring unit can view physiological conditions of a group of first responders in real-time to be sure that none of them are exhibiting adverse physiological symptoms.

In one aspect of the invention, a glove carried sensor can be coupled to a wireless transmitter, carried by the glove. Where the glove includes a pulse oximeter, a variety of hemoglobin derivatives, as would be understood by those of skill in the art, can be sensed including oxygenated hemoglobin or carboxyhemoglobin. Others can also be sensed. All such information can be wirelessly transmitted to the remote monitoring unit and processed to establish the condition of the respective first responder.

In a preferred embodiment, the respective sensor(s) can be located in a ring finger of a glove adjacent to a ring finger of the individual's non-dominant hand. An elastomeric sleeve can be located into the finger cavity of the glove overlying the sensor(s). The ring finger of the individual using the glove extends through the sleeve and is positioned in contact with the sensor.

In one aspect of the invention, the sensor(s) can be attached to the tip of the sleeve adjacent to the end of the finger of the glove. The sleeve advantageously keeps the sensor(s) from rotating or shifting relative to the finger.

In another aspect, a fiber optic cable can be used to couple the sensor(s) to the glove's transmitter. Both the sensor and the transmitter can be energized with a local battery. It will also be understood that each finger of a glove could be equipped with a different sensor, coupled by a cable to the transmitter. In yet another embodiment, multiple sensors can be associated with a selected glove finger, and associated user's finger.

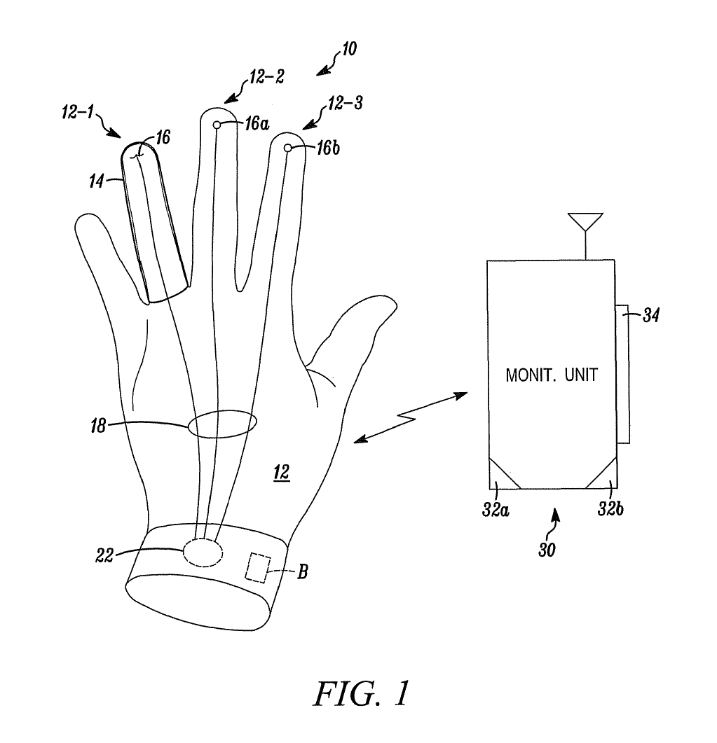

FIG. 1 illustrates an apparatus 10 which includes a protective glove 12 with a ring finger 12-1. An elastic rubber sleeve 14 is positioned in the ring finger 12-1. An oximeter sensor 16, or any other type of physiological sensor, examples noted above, can be attached to an end region of sleeve 14 and also positioned in ring finger 12-1.

An electrical or a fiber optic cable 18, having one strand or multiple communication strands, can couple output electrical signals, from sensor 16 to a local glove carried transmitter 22. Transmitter 22 can be in wireless communication with a displaced monitoring unit 30.

A plurality of sensors such as 16a, 16b can be carried by respective glove fingers, such as 12-2, 12-3 and coupled to the transmitter 22 by one strand or element of the cable 18. If desired multiple sensors such as 16, 16a can be carried in a single glove finger, such as 12-1 without departing from the spirit and scope of the invention.

The unit 30 can include a programmable processor 32a and associated control software 32b. The software 32b can be stored on a computer readable storage medium in the unit 30 for execution by the processor 32a in evaluating results from sensor 16.

Unit 30 can also include a graphical user interface and visual display 34 upon which information from the various glove carried sensor or sensors, such as sensor 16, can be presented and associated with the respective first responder. A battery B can be carried by the glove 12 to energize the above components of the glove.

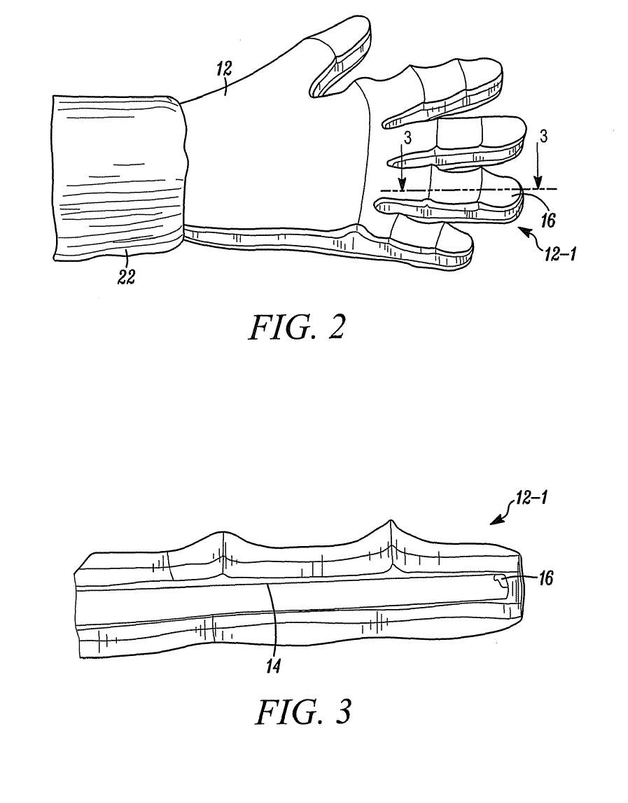

Those of skill will understand that neither the transmission protocol, nor other more specific details of the unit 30 are limitations of the invention. FIGS. 2,3 illustrate additional details of the relationship of the elastomeric rubber sleeve 14 to an attached sensor 16 and the respective finger of the glove 12-1. It will be understood that neither the types of physiological sensors, nor the arrangement of one or more in a selected glove finger are limitations of the invention.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

1. An apparatus comprising:
a multi-finger glove;
at least one elastomeric sleeve position in one finger of the glove; and
at least one physiological sensor carried in the finger of the glove, adjacent to an end thereof.

2. An apparatus as claim 1 where the sensor is selected from a class which includes at least a pulse oximeter, a thermal sensor, a blood pressure sensor and a pulse rate sensor.

3. An apparatus as in claim 2 which includes a transmitter carried by the glove.

4. An apparatus as in claim 3 where the transmitter is coupled to the sensor by a fiber optic link.

5. An apparatus as in claim 4 where the sensor is attached to an end of the sleeve.

6. An apparatus as in claim 5 which includes a displaced monitoring unit in wireless communication with the transmitter.

7. An apparatus as in claim 6 where the monitoring unit includes a computer readable storage medium where information as to sensor output is stored for processing.

8. An apparatus as in claim 7 where the monitoring unit includes a visual display device and the monitoring unit presents on the display device information as to one or more physiological parameters of an individual wearing the glove.

9. An apparatus as in claim 7 with a plurality of gloves in wireless communication with the monitoring unit, each of the gloves carries at least one physiological sensor in wireless communication with the unit via a respective glove carried transmitter.

10. An apparatus as in claim 4 which includes a plurality of physiological sensors carried in one of, a common finger of the glove, or, different fingers of the glove.

11. An apparatus as in claim 10 where at least some of the sensors are attached to a respective elastomeric sleeve.

12. An apparatus as in claim 11 where each of the sensors is coupled to the transmitter.

13. An apparatus as in claim 12 which includes a battery coupled to at least some of the sensors and the transmitter.

14. An apparatus as in claim 9 with a plurality of gloves in wireless communication with the monitoring unit, each of the gloves carries at least one physiological sensor in wireless communication with the unit via a respective glove carried transmitter.

15. An apparatus as in claim 14 which includes at least one glove with a plurality of physiological sensors carried in that glove in one of, a common finger of the glove, or, different fingers of the glove.

16. An apparatus as in claim 15 which includes a plurality of elastomeric sleeves carried in respective fingers of that glove.

17. A multi-finger protective glove comprising:
a glove body from which extends a plurality of protective finger enclosures into which a user can insert a hand and associated fingers;
at least one elastomeric sleeve position in one finger enclosure of the glove, the sleeve has a proximal end and a distal end which abuts an end of the finger enclosure;
at least one physiological sensor selected from a class which includes at least a pulse oximeter, a thermal sensor, a blood pressure sensor and a pulse rate sensor where the at least one sensor is affixed to the elastomeric sleeve adjacent to a selected location in the finger enclosure.

18. A protective glove as in claim 17 where the sensor is affixed to the distal end of the sleeve.

19. A protective glove as in claim 17 which includes a plurality of physiological sensors, at least some of which are carried in respective finger enclosures of the glove.

20. A protective glove as in claim 19 which includes a transmitter, carried by the glove and coupled to the sensors.
